(19) United States
(12) Patent Application Publication
(10) Pub. No.: US 2009/0005795 A1
(43) Pub. Date: Jan. 1, 2009

(54) Surgical needle docking device and method

(76) Inventor: Brandon Giap, San Diego, CA (US)

Correspondence Address: DONN K. HARMS;PATENT & TRADEMARK LAW CENTER, SUITE 100, 12702 VIA CORTINA, DEL MAR, CA, 92014, US

(21) Appl. No.: 11/823,873

(22) Filed: Jun. 28, 2007

(51) Int. Cl. A61B 17/06

(52) U.S. Cl. 606/148

(57) ABSTRACT

A docking device for a suturing needle adapted for engagement to surgical implements. The device has a body formed of substantially soft material pierceable by a pointed end of a needle inserted into it. The needle is held securely in a position for subsequent grasping by a needle holder for a subsequent suture. An axial passage or adhesive provides means for temporary attachment to the surgical implement.

This invention relates to tools and methods used in the suturing of a wound or sewing generally. More specifically, the system relates to a docking system, attachable to surgical implements, by which a needle can be engaged and held static, allowing the user to freely manipulate the needle to a more desirable position for grasping by the fingers or a needle engagement tool. Additionally, the enclosed relates to new methods for using such a device.

### BACKGROUND OF THE INVENTION

Suture techniques are used within the medical community to promote the healing of deep tissue lacerations as well as wounds that are the result of surgery. What is employed almost exclusively in the medical community is a surgical needle with a suture swaged on to the dull end or a hollow end of the needle. This atraumatic needle is preferred to the older method in which a surgical needle had an eye at one end through which the suture was threaded. The prior configuration suffered from a larger profile at the eye end of the needle from the rest of the needle, thus requiring a larger opening to thread the suture through the wound.

Suturing is a very time consuming process and when occurring after surgery it is often necessary to rush the process. The suture process in its most simple form involves a surgical (usually curved) needle, an elongated suture, forceps, and a surgical needle holder. In a first step, the needle is grasped by the needle holder (generally similar to a hemostat) adjacent to the swaged end of the needle (referred to as the loaded position) and driven through the tissue. Subsequent to this first step, the needle is then released and regrasped on the other side of the tissue, at a position adjacent to the sharp tip. So engaged, the needle is then pulled through the tissue, threading the suture through the wound. Meanwhile, forceps are held in the other hand to manipulate tissue during the suture process, and to hold the needle when released by the needle holder.

Once the needle and suture are through the tissue in the first half of a suture, it is necessary to either drop the needle from the needle holder or pass it to an engagement between the tongs of a pair of forceps in order to appropriately grasp the needle to replace it into the loaded position again to repeat the first step. Methods vary from surgeon to surgeon, but the popular mode of temporary docking of the needle is to delicately release the needle holder and concurrently engage it between the small tongs of the forceps. Once so engaged, the surgeon must maintain pressure on the forceps with one hand to hold the needle until re-transferred back to the needle holder. This can be exhausting to the muscles of that hand and can cause dropping of the needle if missed or if the operation requires extensive suturing and the surgeon's hand tires. In some cases, the surgeon simply drops the needle from the holder and onto the patient during this step.

Subsequent to release from the grasp of the needle holder, the surgeon then has to re-grasp the needle with the needle holder into the loaded position yielding an often clumsy and time consuming step in the suture process. This is hardly efficient as the needle is constantly being held and released to the forceps which must be engaged with the other hand, and re-engaged with the needle holder during each stitch. Further time is wasted by straying the instruments and the surgeon's attention from the suture line to where the needle is stored during the changeover. A similar method of suturing is employed in laparoscopic surgery using laparoscopic needle holders since the curved or other needle employed to sew tissue must be pushed from the rear initially, and then pulled through the hole created by the needle. Consequently all such suturing requires a constant change in position by the surgeon from pushing the needle to pulling it.

On some occasions during a suturing session, the surgeon will pass a used needle and suture thread component to other associates in the operating room for disposal. This handling is a common cause of needle sticks which can be very dangerous to the surgeon and operating room personnel. Further, many surgeons handle the needle with their fingers at certain points during the suturing process to change positions of the needle holder or particularly during the first stitch which must be locked with a knot. This too is a common point where needle sticks into the surgeon's fingers can occur.

Thus, there is an unmet need for a device that allows the surgeon to maintain their attention to the suture line during the re-grasping by a needle holder to place the needle at the proper angle and in the loaded position after a stitch. Such a device should provide a receptacle by which to engage and maintain the suture needle in a fixed position, easy for re-grasping and adjacent to the suture line, during the ensuing release and re-engagement procedure with the needle holder. Furthermore, such a device should provide an easy means by which to grasp the needle from its docked position with the device, and to re-engage it with a needle holder such as a hemostat for suturing in any position or angle desired by the surgeon. Additionally, such a device should provide a secure docking of the needle when being passed to associates or being held by the surgeon so as to avoid needle sticks which can pass patient diseases to the surgeon or other personnel.

### SUMMARY OF THE INVENTION

The device herein described and disclosed introduces a docking component which is adapted for attachment to surgical implements commonly employed in medical facilities during the process of suturing on the exterior regions of a patent, or during laparoscopic procedures. The device consists of an elongated soft member which can be frictionally, adhesively, or otherwise engaged to surgical instruments employed during suturing such as a pair of forceps, surgical tweezers, or a retractor. The device provides the surgeon with a docking component for the pointed end of a needle in a semi-soft body portion. This pierced engagement maintains the pointed end of a suturing needle, in a static docking position, when released from a needle holding tool. In this static position, engaged with the docking component, an easy and efficient manner to re-grasp the needle with the needle holder, at the desired angle and position, is provided. Further, an easy and convenient way to store the needle and prevent needle sticks while the surgeon is tying a knot is also provided.

The docking component is formed of a material adapted for temporary engagement with the sharp pointed end of a surgical needle such as silicone or a closed cell foam. The material is of sufficient hardness to accept the needle point piercing it a number of times and to hold it in a biased engagement with the material forming the body. It should be noted that this docking component might also be engaged to the end of an elongated member as a separate surgical instrument. However, the current preferred mode employs means for engagement to existing surgical tools so as not to further crowd the already instrument laden surgical field.

The body forming the docking component is generally elongated thereby providing an extended target for a puncture of the pointed end of the needle. In a preferred mode of the device, the elongated body has a curved sidewall on the side surface adjacent to the suture line. This curved sidewall facing the wound thereby provides an increased area for engagement of the needle at the variety of angles that might be encountered.

In a particularly preferred mode of the device, the body forming the docking component tapers to a smaller cross section as it extends away from the surgeon toward a distal end of the body. This taper reduces the area of the body adjacent to the wound, and provides a better view of the wound and suture line for the surgeon when the body portion is placed adjacent to the wound being sutured.

Means for engagement of the body portion to a surgical instrument is provided in a number of ways. In a first such means for engagement which is particularly preferred for simplicity, an axial cavity is formed through the body portion forming a passage therethrough. This axial cavity has a cross section equal to, or slightly smaller than the diameter of the surgical instrument over which it engages such as a forceps. Since the body portion is made from a soft material such as silicone or a closed cell foam, it has an elastic property allowing the aperture to stretch and engage over the surgical instrument inserted through the passage. This stretching and resulting retraction of the body material once engaged on the surgical instrument provides a biased engagement therewith as the passage collapses back toward the instrument on which it engages.

An alternative means of engagement would be an adhesive material such as a peel and stick configuration adhesive pad. Or, both an aperture and an adhesive means for attachment to a surgical instrument may be provided and one or both employed at the option of the user. In one preferred mode of the device, the body portion may be prepackaged inside the container with the sterile needle and thereby maintained in a sterile state. If only an aperture is provided as a means of engagement, once the device is removed from the package, the surgeon may slide the aperture over the instrument chosen. If adhesive is provided, the device would be adhered to the surface of a chosen instrument. If both the elongated aperture and adhesive options for engagement are provided, the surgeon may either slide the aperture over the surgical instrument of choice, or engage the adhesive on a sidewall to the surgical instrument chosen.

In use, once the body portion is engaged to the surgical instrument, the surgeon is provided with a docking component for temporary removable engagement of the pointed end of the needle once it has been pulled through the wound in the above noted first step. The pointed tip of the needle would be engaged into the body by puncturing it, and held there by the engagement of the tip with the compressed soft body material of the puncture hole.

This engagement thereby positions the aft end of the needle, extending outward from the sidewall on which it engages. This projected aft end positioning allows the surgeon to adjust with great ease and accuracy, the subsequent angle and position at which the needle holding instrument attaches upon the aft end of the needle for the next suture.

The device may also be provided as a separate surgical instrument. The body portion of silicone, closed cell foam, or other substantially soft material, would be engaged to the distal end of an elongated member. In this configuration the device could be employed as a retractor for tissue and docking station for the needle during the transfer of position with the needle holder.

Additionally provided as an optional component is indicia on a top or side surface indicating distance or a measuring scale. For many new surgeons or tired surgeons, placing the sutures in evenly spaced rows can be a hard task to accomplish. Providing a visual means to determine suture distance upon the body portion that is constantly adjacent to the wound, is of great help to some surgeons in maintaining evenly spaced stitches.

In this respect, before explaining at least one embodiment of the invention in detail it is to be understood that the invention is not limited in its application to the details of the construction and to the arrangement of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other methods and systems for carrying out the several purposes of the present invention. It is important, therefore, that the claims be regarded as including such equivalent construction insofar as they do not depart form the spirit and scope of the present invention.

It is an object of this invention to provide a device that can be attached to forceps or other suturing tools or surgical instruments or laparoscopic instruments, by which the suture needle can be engaged in a static docking position when released from a needle holding instrument.

An additional object of this invention is the provision of such a needle docking component which is easily engaged to a variety of surgical instruments.

Yet another object of this invention is the provision of a measurement scale for sutures which may provide the surgeon a visual cue as to their stitch placement.

It is a further object of this invention to provide a method of suturing where the needle is engaged to a docking component and so engaged provides a means of repositioning the needle in the needle holders.

Yet another object of this invention is prevention of needle sticks to the surgeon and associated personnel in the operating room.

These together with other objects and advantages which will become subsequently apparent reside in the details of the construction and method as more fully hereinafter described and claimed, reference being had to the accompanying drawings forming a part thereof, wherein like numerals refer to like parts.

### BRIEF DESCRIPTION OF THE DRAWING FIGURES

FIG. 1 depicts a perspective view of the surgical needle docking device.

FIG. 1a depicts the same view of a particularly preferred shape of docking component with a tapering profile towards a tapered end provides the surgeon with a cleaner line of sight to the suture.

FIG. 2 depicts an end view of the device in FIG. 1 showing the axial aperture communicating through the body.

FIG. 2a depicts an embodiment of the device employing adhesive as a means of engagement to a surgical instrument.

FIG. 2b shows a box that may be employed to dispose of the device and an engaged needle to avoid needle sticks.

FIG. 3a depicts surgical forceps with the suture needle docking device engaged.

FIG. 3b shows a suture site with suture needle being held in the loaded position by needle holders.

FIG. 3c depicts the suture needle being driven through tissue at the suture site.

FIG. 3d shows the needle being regrasped toward its sharp end on the other side of the tissue.

FIG. 3e shows the needle being pulled through the tissue and poised to be docked on the device.

FIG. 3f depicts the suture needle being securely docked to the docking device, allowing easy re-grasping at the desired angle and position.

FIG. 3g shows the needle being held in the loaded position after being easily disengaged from the docking device.

### DETAILED DESCRIPTION OF PREFERRED EMBODIMENT

The disclosed device 10 is for the docking of a suture needle 11 during the suturing of a wound. In one preferred mode of the device 10 there is provided a body 12 portion which serves as a docking component through which an aperture 14 extends axially along the entire length. The body 12 is best formed from soft plastic material such as silicon or closed cell foam or similar materials which allow for easy penetration by the pointed end of a needle and subsequent biased engagement of the needle in the puncture it creates.

The aperture 14 is sized such that it will experience a frictional engagement once engaged onto the forceps 16 or a surgical instrument with an elongated member sized to slide through the aperture 14. The dimension of the aperture 14 should be equal to or slightly smaller than the exterior dimension of the surgical instrument to which it engages through the aperture. If slightly smaller, a biased engagement to the surgical instrument is provided as the material forming the body 12 is stretched and moves back towards it original shape.

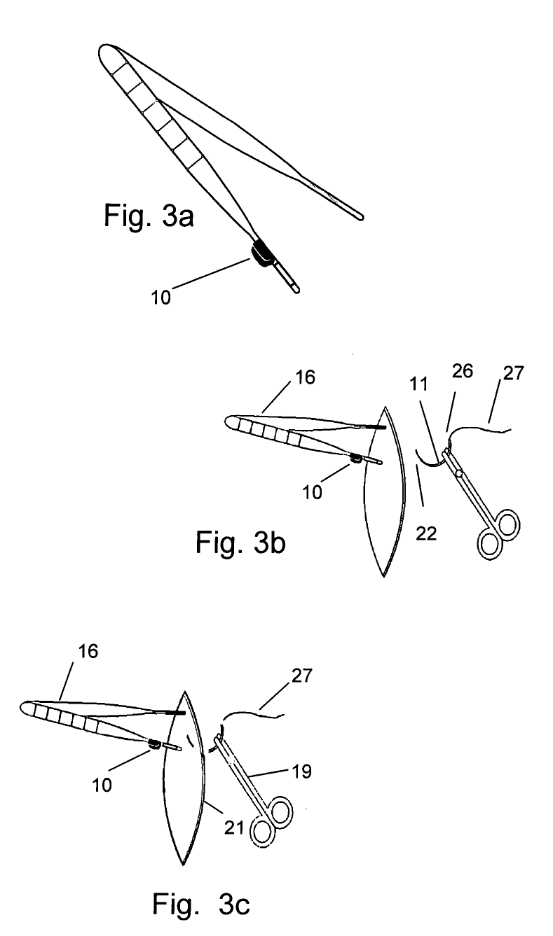

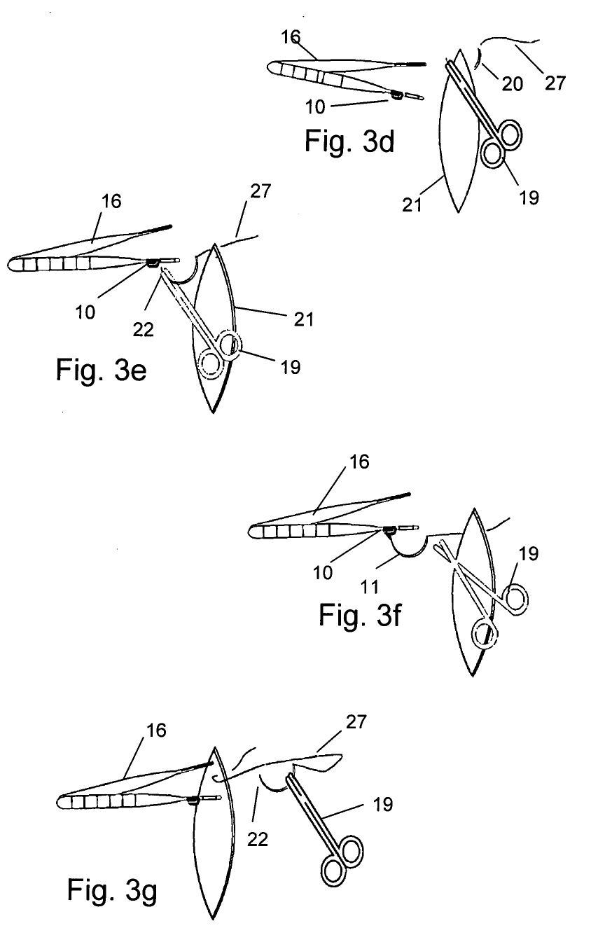

Additionally, when engaging the device 10 to a surgical implement such as forceps 16 or a laparoscopic needle holder, it is particularly preferred to place the aperture 14 forming an axial cavity through the body 12, closer to a planar sidewall 17. In this fashion the forceps 16 will still close at their tip and operate as intended, once the device 10 is slid up a distance on one of the tongs of the forceps 16 as shown in FIG. 3a. Placing the aperture immediately adjacent to a planar sidewall 17 and forming the aperture with parallel interior walls defining an interior circumference, will allow the device to slide onto the forceps 16 or another surgical implement with a substantially planar exterior surface to engage the interior of the aperture 14, and to properly position the long curved sidewall 20 adjacent to the suturing line.

Alternatively, adhesive means for engagement to a surgical instrument such as forceps 16 can be provided in the form of a peel and stick adhesive patch 13 on a sidewall 17. Or, in a particularly easy to engage mode of the device 10 adapted to a mount on virtually any surgical instrument, both the aperture 14 and the adhesive patch 13 may be provided thereby allowing the surgeon a choice of one or a combination of means of attachment from the aperture 14 or adhesive patch 13.

The body 12 in a current preferred mode of the device 10 is formed by two planar sidewalls 15 and 17. These planar sidewalls are joined by a curved sidewall 20. As noted, this curved sidewall 20 provides the surgeon a large area to engage the pointed end 22 of a suturing needle 11 to thereby position the rear or proximal end 26 in a static state extending from the engagement of the tip engaged with the body 12. This engagement of the needle 11 to the body 12 allows the surgeon to rotate the needle 11 or angle it, for an easy re-grasping at the best determined angle relative to the needle holding instrument, for reinsertion through the tissue. Also provided optionally on a side surface of the device such as side 15 is a small gauge 25 for distance measurement which may be employed as a frame of reference or to measure suture spacing. This gauge 25 is best placed on an edge that is easily situated next to the suture line for each reference for the surgeon.

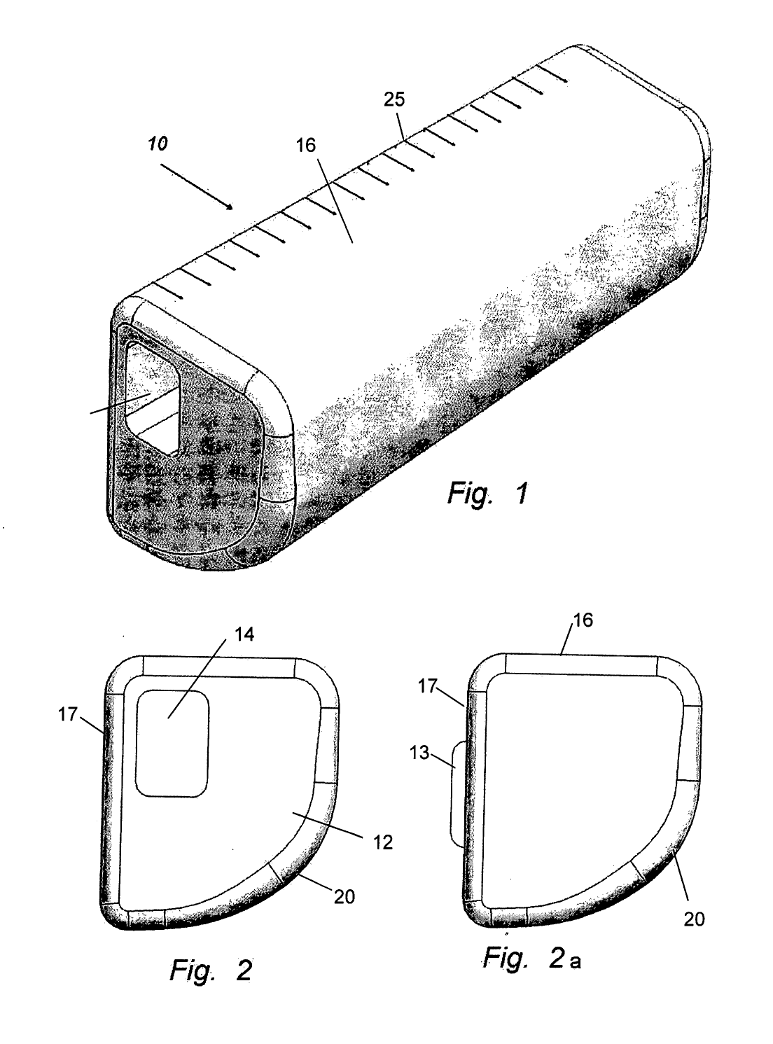

A particularly preferred mode of the device 10 is shown in FIG. 1a which depicts a tapering profile of the body 12 towards a tapered distal end 23. The tapering of the body 12 provides the surgeon with much cleaner line of sight to the suture when the body 12 is placed adjacent thereto. Much like the non-tapered body 12, this mode of the device 10 can be engaged to the surgical instrument using one or a combination of either the aperture 14 or adhesive 13 as convenience or personal choice dictates.

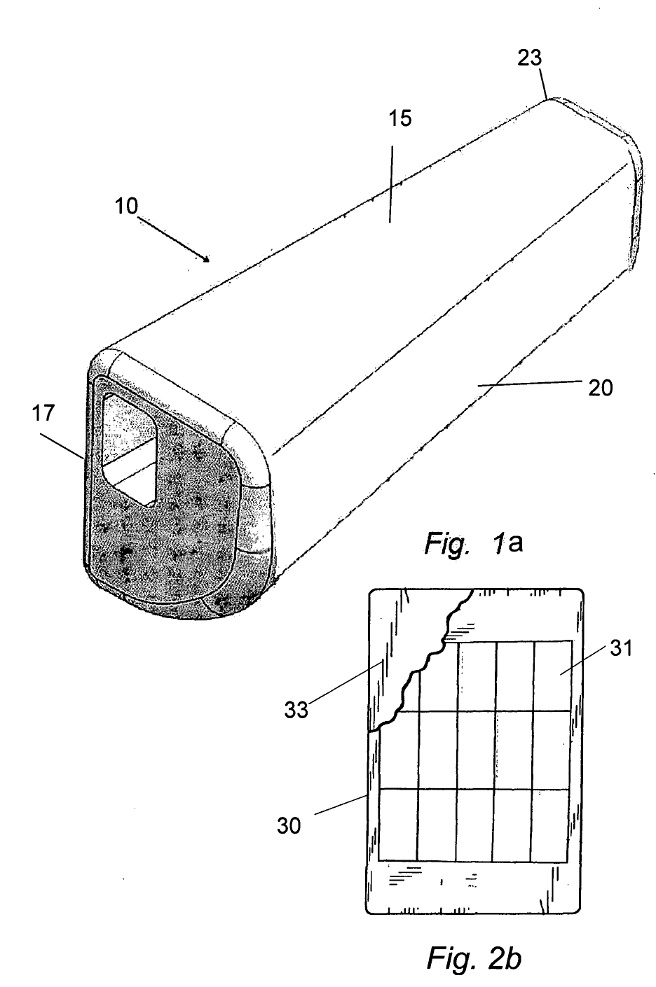

As depicted in FIG. 2b the device 10 can be sold or include with a disposal box 30 having individual cavities 31 inside adapted to hold a needle 11 engaged to a body 12 after its use is ceased. A cover 33 maintains the device and engaged needles in the cavities 31.

FIGS. 3a through 3g depict the common method for employment of the device 10. As seen in FIG. 3a, the device 10 in the as-used position, is frictionally or adhesively engaged to one arm or tong of the forceps 16 or a similar elongated surgical instrument chosen by the surgeon.

In FIG. 3b the suture needle 11 is grasped in the loaded position by the surgical needle holders 19 which as depicted is a hemostat which will lock in a biased frictional engagement to various points on the needle 11. This loaded position is the necessary position for grasping the needle when starting a new stitch since the needle must be pushed through from the rear or proximal end 26 to push the pointed end 22 forward.

FIG. 3c shows the needle 11 being driven through the tissue 21 by the needle holder 19 which is held in the surgeon's hand. Subsequently, as FIG. 3d depicts, once the pointed end 22 of the needle 11 is sufficiently communicated through the tissue 21 for a re-grasping, the surgical needle holders 19 are disengaged from the proximal end 26 of the needle 11. After this disengaging step, the needle holder 19 is re-engaged to the needle 11 adjacent to the pointed end 22 on the other side of the tissue 21. This change in position is required on each stitch since the needle holder 19 cannot pass through the hole formed by the needle 11 thereby requiring the needle 11 to be pulled through the tissue 21 on the second half of the stitch.

FIG. 3e depicts the needle holder 19 being employed to pull the needle 11 from engagement with the tissue 21 with the suture 27 trailing. At this point the needle is poised to be engaged to the body 12 of the device 10 attached to the forceps 16 or other surgical instrument of choice. As noted, it is at this point, that conventionally, surgeons must employ the forceps 16 to grasp the needle during a release from the needle holder, or they can drop the needle somewhere on the patient to be clumsily regrasped. Using the disclosed device and method however, the pointed end 22, at this point in the procedure, is engaged into the body 12 to a docked position. The provided curved sidewall 20 provides a large and easy to engage target for this purpose as best shown in FIG. 3f where the needle 11 is securely docked to the body 12. Once in this docked position, disengagement of the needle 11 from the needle holders 19 may occur leaving the needle 11 with its proximal end 26 suspended above the patient immediately adjacent to the tissue 21 being sutured, and in a position allowing for easy recapture.

As shown in FIG. 3f the protruding proximal end 26 of the needle 11 allows for easy re-grasping by the needle holders 19 in order to secure the needle 11 in the loaded position. Engaged with the pointed end 22 in biased engagement with the body, and with the proximal end so extending, the surgeon can easily re-grasp the needle 11 with the needle holder 19 at any angle relative to the holder 19 to make the next stitch. This re-grasping is shown in FIG. 3g where the needle 11 being held by the needle holders 19 adjacent to the proximal end 26 in the loaded position, poised for the next stitch.

By using the device 10 the surgeon is provided with a much safer and efficient manner to suture a patient without the need to drop the needle 11 in random positions and on surfaces rendering it hard to grasp. By employing the device 10 to maintain the needle 11 elevated and adjacent to the wound to be sutured, no unnecessary time is wasted by a tired surgeon having to struggle to re-grasp the needle 11 into the loaded position in preparation for the next stitch. Employing the device 10 alleviates the need to grasp and hold it with the forceps 16 or to drop the needle 11 altogether and pick it up. Engaged to the body, there is provided an easy access to the proximal end 26 of the needle 11 in order to grasp it with the needle holders 19 in the loaded position, at any angle, and prepared for the next stitch.

Using the device 10 also has the advantages of keeping the surgeon close to the suture site instead of moving back and forth from the suture site to the needle 11 drop site. By maintaining the surgeon's hands adjacent to the suture site at all times, and providing a means by which to re-grasp the needle 11 towards the proximal end 26 without dropping it and having to transfer it back and forth between the forceps 16, the surgeon is able to suture faster and more efficiently. Further, in a laparoscopic procedure, the device 10 provides an internal docking means for the needle which provides the same aforementioned benefits during a suturing process. Additionally, by employing the device 10 the surgeon can easily pass the engaged needle 11 off to a surgical technician, with the pointed end engaged with the body 12, without risk of the needle causing any harm to the surgeon or technician. In fact, the needles 11 can be sold in a system with disposal boxes having interior cavities adapted to hold the needle 11 engaged to the body 12 for disposal so that the pointed end is never exposed to cause a needle stick.

It is to be understood, however, that elements of different construction and configuration and different steps and process procedures and other arrangements thereof, other than those illustrated and described, may be employed for a suturing needle docking device and method of employment thereof, with the spirit of this invention. As such, while the present invention has been described herein with reference to particular embodiments thereof, a latitude of modifications, various changes and substitutions are intended in the foregoing disclosure, and it will be appreciated that in some instance some features of the invention could be employed without a corresponding use of other features without departing from the scope of the invention as set forth in the following claims. All such changes, alterations and modifications as would occur to those skilled in the art are considered to be within the scope of this invention as broadly defined in the appended claims.

What is claimed is:

1. A needle docking apparatus comprising:
a body, said body formed of substantially soft material;
means for attachment of said body to an implement employed during a suturing;
said body having an exterior surface defined by a first end and a second end and an exterior sidewall communicating therebetween; and
said sidewall pierceable by a pointed end of a needle inserted therein in a docked position;
engagement of said body with said pointed end providing means to hold said needle in a position to be grasped for a suture.

2. The needle docking apparatus of claim 1 wherein said means for attachment of said body to an implement comprises one or a combination of a group of means for attachment including an adhesive and an aperture formed in said body.

3. The needle docking apparatus of claim 1 further comprising:
said means for attachment of said body to an implement being a cavity formed in said body, said cavity dimensioned for a frictional engagement upon said implement when inserted therein.

4. The needle docking apparatus of claim 3 wherein said cavity communicates through said body having a dimension determined by an interior circumference; and
said interior circumference being equal to or slightly smaller than the exterior circumference of said implement inserted through said cavity.

5. The needle docking apparatus of claim 4 wherein said implement is a pair of surgical forceps or tweezers having a portion thereof inserted through said cavity.

6. The needle docking apparatus of claim 1 further comprising:
said body having a cross sectional area which tapers from a wider portion at said first end to a narrower portion at said second end.

7. The needle docking apparatus of claim 2 further comprising:
said body having a cross sectional area which tapers from a wider portion at said first end to a narrower portion at said second end.

8. The needle docking apparatus of claim 3 further comprising:
said body having a cross sectional area which tapers from a wider portion at said first end to a narrower portion at said second end.

9. The needle docking apparatus of claim 4 further comprising:
said body having a cross sectional area which tapers from a wider portion at said first end to a narrower portion at said second end.

10. The needle docking apparatus of claim 5 further comprising:
said body having a cross sectional area which tapers from a wider portion at said first end to a narrower portion at said second end.

11. The needle docking apparatus of claim 4 wherein:
said cavity communicates axially through said body in a position off-center in a closer proximity to a first portion of said sidewall on a first side of said body.

12. The needle docking apparatus of claim 11 wherein:
said first portion of said sidewall is substantially planar and is opposite a second portion of said sidewall having a curved portion.

13. The needle docking apparatus of claim 1 wherein:
a first portion of said sidewall is substantially planar and a second portion of said sidewall is substantially curved; and
said means for attachment being adhesive positioned on said first sidewall.

14. The needle docking apparatus of claim 12 additionally comprising:
a third portion of said sidewall communicating between said first portion and said second portion; and
said third portion being substantially planar.

15. The needle docking apparatus of claim 13 additionally comprising:
a third portion of said sidewall communicating between said first portion and said second portion; and
said third portion being substantially planar.

16. The needle docking apparatus of claim 14 additionally comprising:
indicia located on said third portion of said sidewall, said indicia defining a distance scale.

17. The needle docking apparatus of claim 15 additionally comprising:
indicia located on said third portion of said sidewall, said indicia defining a distance scale.

18. The needle docking apparatus of claim 1 additionally comprising:
a container having at least one cavity therein; and
said cavity sized to accommodate deposit therein of said body while in engagement with said pointed end of said needle whereby said needle may be disposed into said cavity while engaged with said pointed end as a means to prevent needle punctures to users.

19. A method for surgical suturing using needle docking component having a body portion adapted for puncture by the pointed end of a suturing needle and engageable with a surgical implement, comprising:
a) engaging said body of said needle docking component with a surgical implement;
b) engaging said pointed end of said suturing needle, into a punctured engagement with said body to a docked position; and
c) grasping said needle between said pointed end and a distal end opposite from said pointed end and removing it from said docked position for an engagement of a stitch;

20. The method of claim 19 further comprising:
d) after each such engagement of a said stitch, repeating steps a through c until said suturing with said needle is finished;
e) disposing of said needle and said body while said needle is in said docked position with said body.
